(19) United States
(12) Patent Application Publication
(10) Pub. No.: US 2008/0031408 A1
(43) Pub. Date: Feb. 7, 2008

(54) QUANTIFICATION OF SINUS PROBLEMS IN A PATIENT

(76) Inventors: Predrag Sukovic, Birmingham, MI (US); Neal Clinthorne, Ann Arbor, MI (US); Joseph Webster Stayman, Ann Arbor, MI (US); Miodrag Rakic, Redondo Beach, CA (US)

Correspondence Address: CARLSON, GASKEY & OLDS, P.C., 400 WEST MAPLE ROAD, SUITE 350, BIRMINGHAM, MI, 48009, US

(21) Appl. No.: 11/828,604

(22) Filed: Jul. 26, 2007

Related U.S. Application Data
(60) Provisional application No. 60/836,118, filed on Aug. 7, 2006.

(51) Int. Cl. A61B 6/03; A61B 6/00

(52) U.S. Cl. 378/15; 378/4; 600/407

(57) ABSTRACT

A CT scanner takes a three-dimensional CT image, and a right sinus cavity and a left sinus cavity of a patient are identified in the CT image. The sinus cavities can be manually or automatically identified. When the sinus cavities are clear, the sinus cavities appear black in the CT image. When fluid or static polyps fill the sinus cavities, the sinus cavities appear gray in the CT image. A computer evaluates the CT image to quantify the amount of fluid or static polyps in the sinus cavities.

### REFERENCE TO RELATED APPLICATION

This application claims priority to U.S. Provisional Patent Application No. 60/836,118 filed Aug. 7, 2006.

### BACKGROUND OF THE INVENTION

The present invention relates generally to a CT scanner that quantifies sinus problems in a patient.

A CT scanner takes a plurality of x-ray images of a part of a patient to create a three dimensional CT image that can provide a visual image of the patient's sinus cavities. A technician can visually review the CT image to determine the condition of the sinus cavities and the presence of any fluid or static polyps in the sinus cavities. However, the technician cannot quantify a sinus condition of the patient based on the visual evaluation of the CT image. This makes evaluating the progression of a problem or a treatment difficult to assess.

Hence, there is a need in the art for a CT scanner that quantifies sinus problems in a patient to determine a sinus condition.

### SUMMARY OF THE INVENTION

A CT scanner includes a gantry that supports and houses components of the CT scanner. The gantry includes a cross-bar section, and a first arm and a second arm each extend substantially perpendicularly from opposing ends of the cross-bar section. The first arm houses an x-ray source that generate x-rays. The second arm houses a complementary flat-panel detector. The x-rays are directed toward the detector, which includes a converter that converts the x-rays from the x-ray source to visible light and an array of photodetectors behind the converter to create an image. As the gantry rotates about the patient, the detector takes a plurality of x-ray images at a plurality of rotational positions.

A CT image of a head of a patient shows a right sinus cavity and a left sinus cavity. When the sinus cavities are clear and filled with air, the sinus cavities appear black in the CT image. When the sinus cavities are filed with fluid or static polyps, the sinus cavities appear gray in the CT image. A computer evaluates the CT image and identifies the location of the sinus cavities.

In one example, the computer automatically identifies the location of the sinus cavities by density. The density of the bones in the CT image is greater than the density of the soft tissue and the sinus cavities in the CT image. The computer locates the sinus cavities based on the lowest density regions of the CT image.

In another example, a technician manually identifies the location of the sinus cavities by visually evaluating the CT image on a display. The technician uses a mouse and a keyboard of the computer to indicate the location of the sinus cavities on the CT image.

After the location of the sinus cavities is identified, the computer evaluates the CT image to quantify the amount of fluid or static polyps in the sinus cavities. If the sinus cavities contain fluid or static polyps, the computer quantifies the amount of fluid or static polyps in the sinus cavities based on the amount of gray in the space defined as the sinus cavities. Based on the amount of gray, the technician can evaluate the sinus cavities to determine if the patient has a sinus condition or monitor the progress of a patient with a sinus condition.

### BRIEF DESCRIPTION OF THE DRAWINGS

FIG. 1 illustrates a first embodiment of a CT scanner of the present invention;

FIG. 2 illustrates the CT scanner of FIG. 1 with a part of a patient received in the CT scanner;

FIG. 3 illustrates a second embodiment of the CT scanner of the present invention;

FIG. 4 illustrates a computer employed with the CT scanner of the present invention;

FIG. 5 illustrates a CT image of a patient taken when the sinus cavities of the patient are clear; and

FIG. 6 illustrates a CT image of the patient of FIG. 5 taken when the sinus cavities of the patient are filled with fluid or static polyps.

### DETAILED DESCRIPTION OF THE PREFERRED EMBODIMENT

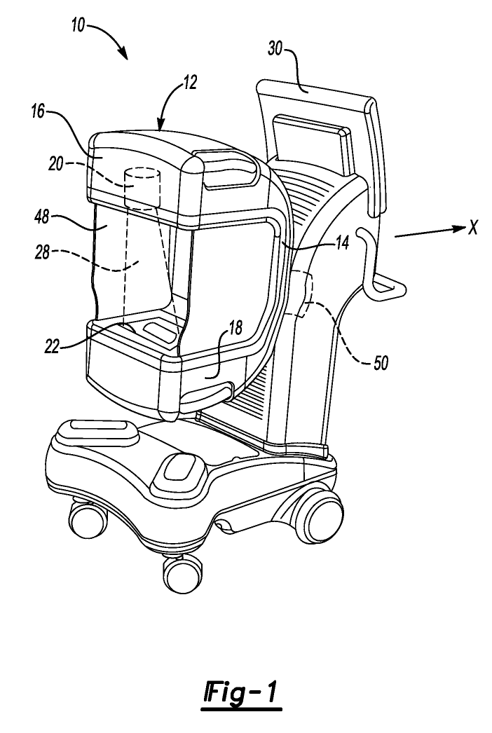

FIG. 1 illustrates a CT scanner 10 of the present invention. The CT scanner 10 includes a gantry 12 that supports and houses components of the CT scanner 10. In one example, the gantry 12 includes a cross-bar section 14, and a first arm 16 and a second arm 18 each extend substantially perpendicularly from opposing ends of the cross-bar section 14 to form the c-shaped gantry 12. The first arm 16 houses an x-ray source 20 that generate x-rays 28. In one example, the x-ray source 20 is a cone-beam x-ray source. The second arm 18 houses a complementary flat-panel detector 22. The x-rays 28 are directed toward the detector 22 which includes a converter (not shown) that converts the x-rays 28 from the x-ray source 20 to visible light and an array of photodetectors behind the converter to create an image. As the gantry 12 rotates about the patient P, the detector 22 takes a plurality of x-ray images at a plurality of rotational positions. Various configurations and types of x-ray sources 20 and detectors 22 can be utilized, and the invention is largely independent of the specific technology used for the CT scanner 10.

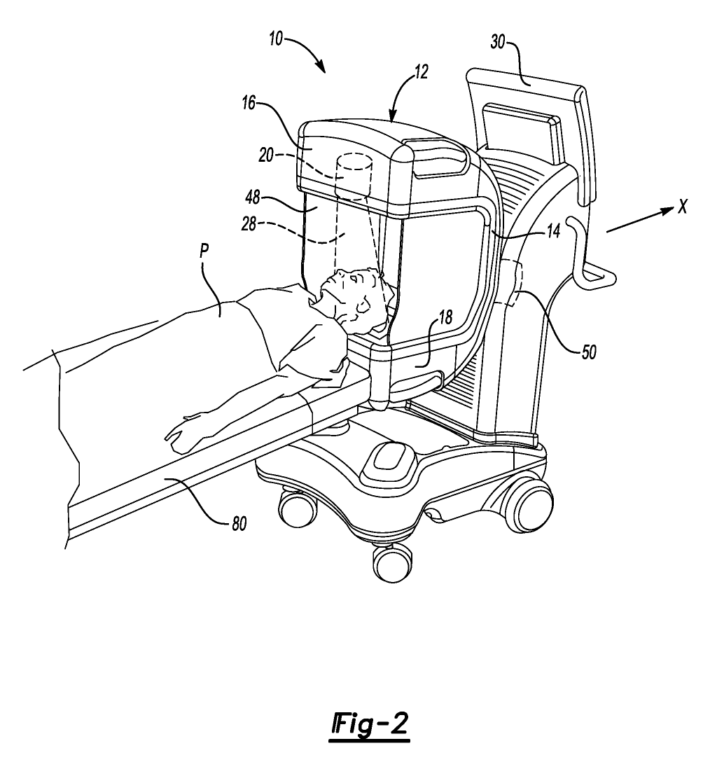

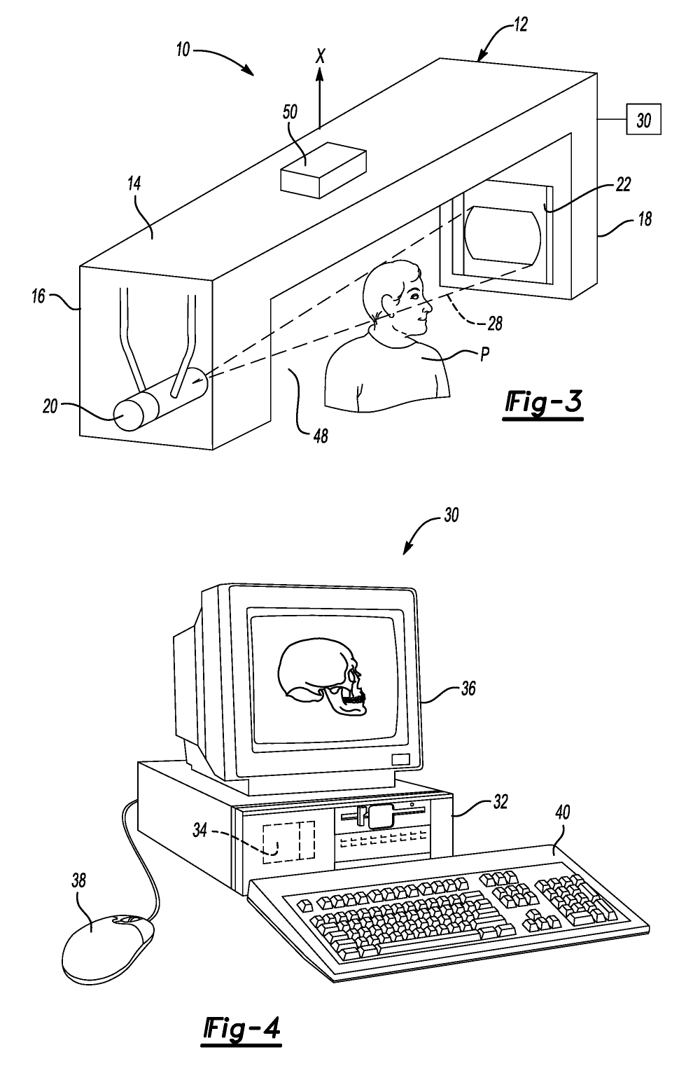

FIG. 2 illustrates the CT scanner 10 with a part of the patient P received in a space 48 between the first arm 16 and the second arm 18. A motor 50 rotates the gantry 12 about an axis of rotation X to obtain a plurality of x-ray images of the patient P at the plurality of rotational positions. The gantry 12 can be rotated approximately slightly more than 360 degrees about the axis of rotation X. In one example, as shown in FIGS. 1 and 2, the axis of rotation X is substantially horizontal. In this example, the patient P is typically lying down on a table 80. Alternatively, as shown in FIG. 3, the axis of rotation X is substantially vertical. Typically, in this example, the patient P is sitting upright.

As shown schematically in FIG. 4, the CT scanner 10 further includes a computer 30 having a microprocessor or CPU 32, a storage 34 (memory, hard drive, optical, and/or magnetic, etc), a display 36, a mouse 38, a keyboard 40 and other hardware and software for performing the functions described herein. The computer 30 powers and controls the x-ray source 20 and the motor 50. The plurality of x-ray images taken by the detector 22 are sent to the computer 30. The computer 30 generates a three-dimensional CT image from the plurality of x-ray images utilizing any known techniques and algorithms. The three-dimensional CT image is stored on the storage 34 of the computer 30 and can be displayed on the display 36 for viewing.

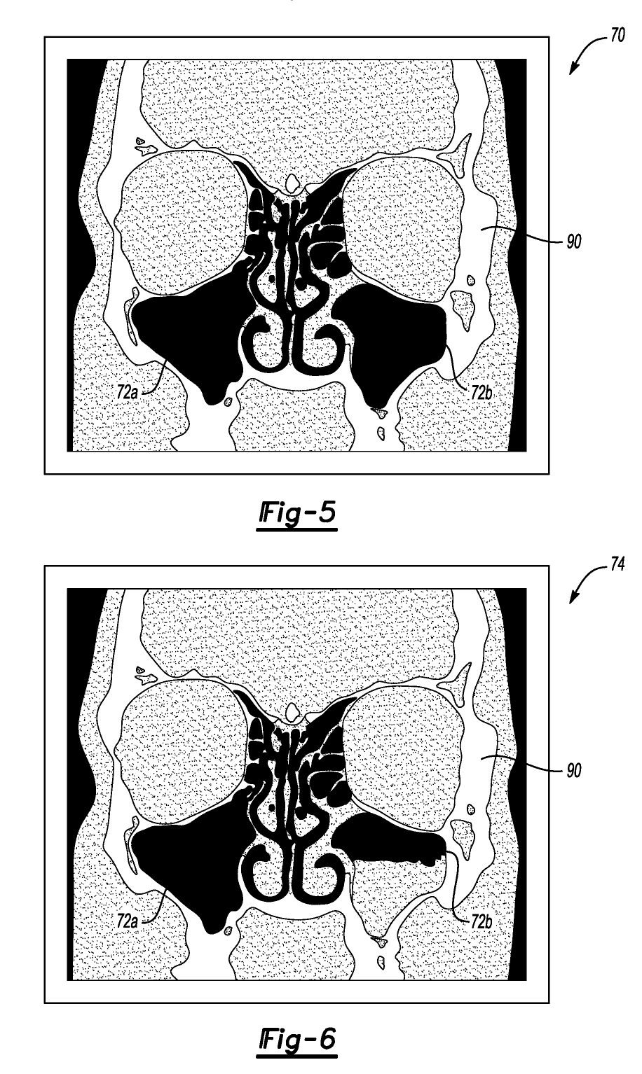

FIG. 5 illustrates a two dimensional CT image 70 of a head of a patient P. The CT image 70 includes a right sinus cavity 72a and a left sinus cavity 72b. Both the sinus cavities 72a and 72b are filled with air and clear, and therefore free of fluid or static polyps. The sinus cavities 72a and 72b appear black on the CT image 70. The bones 90 of the patient P appear white in the CT image 70.

FIG. 6 shows a two dimensional CT image 74 of the same patient P. The right sinus cavity 72a is clear and appears black in the CT image 74. However, the left sinus cavity 72b is filled with either fluid or static polyps and appears gray in the CT image 74.

In one example, software is installed on the computer 30 to evaluate the CT images 70 and 74 and to identify the location of the sinus cavities 72a and 72b in the CT images 70 and 74. In one example, the computer 30 automatically identifies the location of the sinus cavities 72a and 72b by density. The density of the bones 90 is greater than the density of the soft tissue and the sinus cavities 72a and 72b. The computer 30 locates the sinus cavities 72a and 72b based on the lowest density regions of the CT images 70 and 74.

In another example, a technician manually identifies the location of the sinus cavities 72a and 72b by visually evaluating the CT images 70 and 74 on the display 36. In this example, the technician uses the mouse 38 and the keyboard 40 of the computer 30 to define the location of the sinus cavities 72a and 72b on the CT images 70 and 74.

After the location of the sinus cavities 72a and 72b is identified in the CT images 70 and 74, the computer 30 evaluates the CT images 70 and 74, specifically the sinus cavities 72a and 72b in the CT images 70 and 74, to quantify the amount of fluid or static polyps, if any, in the sinus cavities 72a and 72b. Preferably, the sinus cavities 72a and 72b should be free of fluid and static polyps. If the sinus cavities 72a and 72b contain fluid or static polyps, the computer 30 quantifies the amount of fluid or static polyps in the sinus cavities 72a and 72b.

For example, the computer 30 can determine what percentage of the sinus cavities 72a and 72b are filled with fluid or static polyps based on the amount of gray in the space defined as the sinus cavities 72a and 72b. That is, the amount of gray in the sinus cavities 72a and 72b represents the amount of fluid or static polyps in the sinus cavities 72a and 72b. The greater the percentage of gray in the sinus cavities 72a and 82b, the greater the amount of fluid or static polyps in the sinus cavities 72a and 72b. Based on the percentage of gray, the amount of fluid or static polyps in the sinus cavities 72a and 72b can be quantified. This allows the sinus cavities 72a and 72b to be evaluated to determine if the patient P has a sinus condition or monitor the progress of a patient P with a sinus condition.

The foregoing description is only exemplary of the principles of the invention. Many modifications and variations are possible in light off the above teachings. It is, therefore, to be understood that within the scope of the appended claims, the invention may be practiced otherwise than using the example embodiments which have been specifically described. For that reason the following claims should be studied to determine the true scope and content of this invention.

1. A method of quantifying a sinus condition of at least one sinus cavity of a patient, the method comprising the steps of:
generating an image of the patient;
locating the at least one sinus cavity in the image; and
quantifying the sinus condition of the at least one sinus cavity based on the image.

2. The method as recited in claim 1 wherein the image is a three dimensional CT image, and the step of generating the image of the patient includes generating x-rays, taking a plurality of two dimensional x-rays images of the patient and generating the three dimensional CT image with the plurality of two dimensional x-ray images.

3. The method as recited in claim 2 further including the step of positioning a part of the patient in a CT scanner, rotating a gantry of the CT scanner about an axis of rotation, and taking the plurality of two dimensional x-ray images during the step of rotating the gantry.

4. The method as recited in claim 1 wherein the at least one sinus cavity appears black in the image when the at least one sinus cavity is clear and the at least one sinus cavity appears gray in the image when the at least one sinus cavity is filled with at least one of fluid and a static polyp.

5. The method as recited in claim 1 wherein the step of locating the at least one sinus cavity includes automatically locating the at least one sinus cavity in the image.

6. The method as recited in claim 5 wherein the step of automatically locating the at least one sinus cavity includes locating the at least one sinus cavity based on a lowest density region in the image.

7. The method as recited in claim 1 wherein the step of locating the at least one sinus cavity includes manually locating the at least one sinus cavity in the image.

8. The method as recited in claim 7 wherein the step of manually locating the at least one sinus cavity includes visually identifying the at least one sinus cavity in the image on a display and employing at least one of a mouse and a keyboard to identify the at least one sinus cavity in the image.

9. The method as recited in claim 1 wherein the step of quantifying the sinus condition includes quantifying an amount of at least one of fluid and a static polyp in the at least one sinus cavity.

10. The method as recited in claim 1 wherein the step of quantifying the sinus condition includes determining an amount of gray in the at least one sinus cavity in the image, and the amount of gray represents an amount of at least one of fluid and a static polyp in the at least one sinus cavity.

11. The method as recited in claim 10 wherein the amount of the at least one of fluid and a static polyp in the at least one sinus cavity increases as the amount of gray in the at least one sinus cavity in the image increases.

12. A CT scanner comprising:
an x-ray source to generate x-rays;
an x-ray detector mounted opposite the x-ray source; and
a computer to generate an image of the patient, to locate at least one sinus cavity in the image, and to quantify a sinus condition of the at least one sinus cavity of the patient based on the image.

13. The CT scanner as recited in claim 12 wherein the CT scanner takes a plurality of two dimensional x-rays images of the patient and the computer generates a three dimensional CT image with the plurality of two dimensional x-ray images.

14. The CT scanner as recited in claim 12 further including a gantry including a cross-bar section, a first arm and a second arm that each extend substantially perpendicularly to the cross-bar section, wherein the x-ray source is housed in the first arm and the x-ray detector is housed in the second arm.

15. The CT scanner as recited in claim 14 further including a motor to rotate the gantry about an axis of rotation while the CT scanner takes the plurality of two dimensional x-ray images.

16. The CT scanner as recited in claim 12 wherein the x-ray source is a cone-beam x-ray source.

17. The CT scanner as recited in claim 12 wherein the at least one sinus cavity appears black in the image when the at least one sinus cavity is clear and the at least one sinus cavity appears gray in the image when the at least one sinus cavity is filled with at least one of fluid and a static polyp.

18. The CT scanner as recited in claim 12 wherein the computer automatically locates the at least one sinus cavity in the image.

19. The CT scanner as recited in 18 wherein the computer automatically locates the at least one sinus cavity in the image based on a lowest density region.

20. The CT scanner as recited in claim 12 wherein an operator manually locates the at least one sinus cavity in the image.

21. The CT scanner as recited in claim 20 wherein the operator manually locates the at least one sinus cavity in the image using at least one of a keyboard and a mouse.

22. The CT scanner as recited in claim 12 wherein the computer quantifies the sinus condition by quantifying an amount of at least one of fluid and a static polyp in the at least one sinus cavity.

23. The CT scanner as recited in claim 12 wherein the computer quantifies the sinus condition by determining an amount of gray in the at least one sinus cavity in the image, and the amount of gray represents an amount of at least one of fluid and a static polyp in the at least one sinus cavity.

24. The CT scanner as recited in claim 23 wherein the amount of gray in the at least one sinus cavity in the image increases as the amount of the at least one of fluid and a static polyp in the at least one cavity increases.
